(19) United States
(12) Patent Application Publication
(10) Pub. No.: US 2006/0094657 A1
(43) Pub. Date: May 4, 2006

(54) Inhibiting development of microvessels within coronary or peripheral vessel walls for restenosis/atherosclerosis prevention or therapy

(75) Inventors: Stephen E. Epstein, Rockville, MD (US); Ran Kornowski, Rockville, MD (US); Shmuel Fuchs, Rockville, MD (US); Martin Leon, Bethesda, MD (US)

Correspondence Address: FROMMER LAWRENCE & HAUG, 745 FIFTH AVENUE- 10TH FL., NEW YORK, NY, 10151, US

(73) Assignee: Cardiovascular Research Foundation

(21) Appl. No.: 11/295,095

(22) Filed: Dec. 6, 2005

Related U.S. Application Data
(63) Continuation of application No. 09/478,621, filed on Jan. 5, 2000, now abandoned.
(60) Provisional application No. 60/115,977, filed on Jan. 15, 1999.

(51) Int. Cl. A61K 38/17; A61K 48/00

(52) U.S. Cl. 514/12; 514/44; 424/93.2

(57) ABSTRACT

Disclosed and claimed are compositions and methods for therapy and/or prevention of restenosis and/or atherosclerosis. The compositions can include an agent for inhibiting VEGF and an agent for including vessel maturation; for instance, the soluble VEGF receptor and ang-1. Embodiments can include kits.

### RELATED APPLICATIONS

This application claims priority from U.S. application Ser. No. 60/115,977, filed Jan. 15, 1999; and, that application and all documents cited therein, and all documents cited or referenced in documents cited in that application, are hereby incorporated herein by reference.

### FIELD OF THE INVENTION

The present invention relates to compositions and methods for the preventing and/or treatment of restenosis and/or atherosclerosis.

The present invention further relates to compositions and methods for inhibiting the development of microvessels within the wall of coronary and/or peripheral vessels.

Microvessels can develop in response to angioplasty procedures and/or stent implantation, and develop during the development of atherosclerosis; and thus, the present invention relates to compositions and methods for inhibiting the development of microvessels within the wall of coronary and/or peripheral vessels in response to angioplasty procedures and/or stent implantation, and during the development of atherosclerosis

The present invention further relates to compositions and methods for inhibiting the development of microvessels within the wall of coronary and/or peripheral vessels in response to angioplasty procedures and/or stent implantation, and during the development of atherosclerosis, for preventing and/or treating restenosis and/or atherosclerosis.

The present invention further relates to compositions and methods containing or employing agents having anti-angiogenic effects, such as endostatin, angiostatin, thallidamide or other agents which either bind to the angiogenic agent or to its receptor or by inhibiting any aspect of the signaling cascade initiated by the binding of the angiogenic ligand to its receptor. The agent can be a protein or a gene; for instance a gene which expresses a protein in vivo; the gene could be delivered by a vector, e.g. plasmid or viral vector; and, targets of anti-angiogenic strategies can include VEGF and/or its receptors and/or its signalling cascade, bFGF and/or its receptors and/or its signalling cascade, any of the other members of the family of FGFs and their signalling cascades, angiopoeitin-1 (ang-1) and/or its receptor and/or its signalling cascade, angiopoeitin-2 (ang-2) and/or its receptor and/or its signalling cascade.

The present invention also relates to any or all of: microvascular angiogenesis (expansion of the vasovasorum) occurring during both atherogenesis and during restenosis; expression of VEGF and ang-1, e.g., coordinated sequential expression of VEGF and ang-1, with activation of their signaling cascades, which are consistent components of post-embryonic microvascular angiogenic processes that occur during restenosis and atherosclerosis; upregulation of VEGF, which is necessary for the angiogenic process; and upregulation of either ang-1 and/or ang-2, which are necessary for the induction and maturation of new vessels, and upregulation of members of the family of FGFs and their signalling cascades. Accordingly, the present invention relates to methods and compositions for inhibiting VEGF, e.g., the soluble VEGF receptor, for inhibiting expression of VEGF and/or VEGF activity, for inducing ang-1, or for inhibiting ang-2 and/or inhibiting members of the family of FGFs and their signalling cascades, that is, methods and compositions to reduce microangiogenesis and/or inhibit atherosclerosis and/or restenosis.

The present invention further relates to methods and compositions for administering an agent which inhibits VEGF, e.g., the soluble VEGF receptor, and which inhibits the family of FGFs and their signalling cascades.

The present invention also relates to methods and compositions for administering an agent which induces vessel maturation, and which thereby may inhibit the development of vessel sprouting and thereby the development of new vessels e.g., ang-1.

The present invention yet further relates to methods and compositions for administering an agent which inhibits VEGF, e.g., the soluble VEGF receptor, which inhibits the family of FGFs and their signalling cascades, and an agent which induces vessel maturation, e.g., ang-1. The administration can be sequential, simultaneous, or separated by a desired time period and can be by any suitable means.

Accordingly, the present invention relates to protein delivery, including by in vivo expression methods, to prevent or treat restenosis and/or atherosclerosis. The present invention relates to such protein delivery to inhibit the development of microvessels (vasovasorum). The present invention relates to such protein delivery for anti-angiogenesis, e.g., to suppress angiogenesis; for instance, to thereby inhibit or prevent or prolong the onset of restenosis and/or atherosclerosis.

Various documents are cited in the following text, or in a reference section preceding the claims. Each of the documents cited herein, and each of the references cited in each of those various documents, is hereby incorporated herein by reference. None of the documents cited in the following text is admitted to be prior art with respect to the present invention.

### BACKGROUND OF THE INVENTION

As discussed generally by Jean Marx at page 320 of Science, Vol. 265 (Jul. 15, 1994), each year about 330,000 patients in the United States undergo coronary and/or peripheral angioplasty, a procedure designed to open up blood vessels, e.g., coronary arteries, clogged by dangerous atherosclerotic plaques (atherosclerosis) and thereby restore normal blood flow. For a majority of these patients, the procedure works as intended. Nearly 33% of these patients (and maybe more by some accounts), however, develop restenosis, wherein the treated arteries become quickly clogged again. These patients are no better off, and sometimes worse off, than they were before angioplasty. Excessive proliferation of smooth muscle cells in blood vessel walls contributes to restenosis.

While the use of stents has appreciably reduced the rate of restenosis, even with this treatment, restenosis occurs in 5 to 20% of patients. Thus, the problem of restenosis is formidable, despite recent advances in reducing its incidence.

Two primary mechanisms appear to be involved in the development of restenosis.

First, recoil of the vessel wall (negative remodeling) leads to gradual narrowing of the vessel lumen.

Second, an exaggerated healing response of medial and/or adventitial smooth muscle cells (SMCs) to vascular injury, which involves the excessive proliferation of SMCs and the migration of SMCs to the subintima, where they continue to proliferate and begin to secrete extracellular matrix.

These processes involving SMCs cause the neointimal mass to expand and gradually encroach upon the coronary lumen; ultimately the expanding lesion narrows the vessel, increases resistance to blood flow, and causes ischemic symptoms.

In the absence of stenting, both remodeling and an expanding neointima contribute to restenosis; when stents are deployed negative vascular remodeling is prevented and restenosis occurs only as a result of the expanding neointimal mass.

Given these pathophysiologic mechanisms, the problem of controlling restenosis becomes largely the problem of controlling the development of the neointimal mass and, in the absence of stenting, also in controlling the amount of negative vascular remodeling.

The potential role of the vasovasorum as a determinant of atherosclerotic plaque mass was first raised by the studies of Barger et al., 1984 (Barger et al., “Hypothesis: vasavasorum and neovascularization of human coronary arteries. A possible role in the pathophysiology of atherosclerosis.” N Eng J Med 310(3): 175-7(January 1984)). These investigators demonstrated that atherosclerotic plaques were highly vascularized. In particular, the mass of vasovasorum microvessels present in the wall of the coronary artery at the site of atherosclerotic plaque was found to be increased roughly in proportion to plaque mass.

This interesting observation could not however, distinguish between two alternative possibilities: 1) that the angiogenic stimulus derived in some way from the growing plaque, versus 2) that increasing angiogenesis causes plaque growth. The latter possibility was suggested to play a role by the recent study from Folkman's laboratory.

Dr. Folkman and his colleagues demonstrated that in apoE knockout mice, which are prone to develop atherosclerosis, treatment with endostatin (a potent anti-angiogenic drug) reduces the magnitude of plaque mass development. This finding was accompanied by a decrease in the number of blood vessels supplying the plaque. Thus, these results are compatible with the concept that atherosclerotic plaque growth is limited by its blood supply, and therefore determined by angiogenesis processes involving the vasovasorum. Dr. Folkman published an abstract of the work in the November 1998 edition of Circulation, and presented his findings at the TCT Symposium in Washington in October 1998, and at the American Heart Association Annual Meeting in Dallas, in November 1998.

Angiogenesis involves the sprouting of capillaries from preexisting blood vessels and/or the development of new vessels. This process is controlled by the action of several angiogenic growth factors and their tyrosine kinase receptors. Currently, the basic mechanisms responsible for angiogenesis are not fully understood.

However, two systems involving vascular endothelial growth factor (VEGF) and the angiopoietin-1/angiopoietin-2 ligands, along with their specific receptors (VEGF-R1, VEGF-R2 and Tie-2 respectively), seem to seem to have a unique and specific roll in the induction and maintenance of new blood vessel formation. Studies in mice carrying homozygous disruption in these receptors have demonstrated VEGF and angopoietin-1 act in sequence; 1) VEGF, through VEGF-R1, induces endothelial cell-cell interaction, proliferation, and tube formation; 2) angiopoietin-1, through binding to its receptor Tie-2, elicits recruitment and interaction with peri-endothelial support cells, thus maintaining vessel integrity and stabilizing newly formed blood vessels. Angiopoietin-2 appears to be a functional antagonist of antiopoietin-1, and its expression may be a necessary step in destabilizing an existing vessel, thereby allowing it to initiate new vascular buds and branches.

Accordingly, improvements in the therapy, prophylaxis and diagnosis of restenosis and/or atherosclerosis, especially in compositions therefor and methods thereof, would be an advance over the state of the art.

Reference is made to WO 98/33510, Kwon et al., Journal of Clinical Investigation, 101(8): 1551-56(April 1998), Kwon et al., “Adventitial Vasa Vasorum in Balloon-injured Coronary Arteries: Visualization and Quantitation by a Microscopic Three-dimensional Computed Tomography Technique,” J. Am. Coll. Cardiol. (1998), Inoue et al., Circulation 98:2108-2116(November 1998), and Asahara et al., Circ Res 83(3):233-240(August 1998).

WO 98/33510 relates to restenosis and/or atherosclerosis diagnosis, prevention and therapy, e.g., by decreasing viral load; and, the reader is respectfully directed to that document for information and literature citations involving restenosis and/or atherosclerosis diagnosis, prevention and therapy. The Kwon et al articles provide a visualization and quantitation of three-dimensional spacial patterns of vaso vasorum in normal and balloon injured or hypercholesterolemic porcine coronary arteries. Asahara et al. relates to the effects of angiopoietin on postnatal neovascularization. And, Inoue et al. is directed to VEGF expression in atherosclerotic lesions.

Further, mention is made of Takahashi et al. Jpn J Cancer Res 89(4):445-51(April 1998) which may relate to clotrimazole, an imidazole antimycotic, as an inhibitor of angiogenesis and Raymond Presse Med 27(24):1221-4(July 1998) which discusses antiangiogenic properties of endostatin and angiostatin, and cites Folkman, Nature 390:404-7(1997). These documents do not appear to directly address inhibiting VEGF and/or inducing vessel maturation, for instance, for preventing or treating restenosis and/or atherosclerosis, in contrast to the present invention.

Accordingly, in general, as a contrast to the foregoing documents and those cited otherwise herein and that which is believed to have been the knowledge in the art, the present invention addresses restenosis and/or atherosclerosis prevention and/or therapy by inhibiting the specific ligands, receptors, and/or their signalling cascades that have been identified as the natural pathways by which new vessels develop. Thus, the present invention inhibits microvessel development; for instance, by inhibiting VEGF or its activity or its receptors and/or by inducing vessel maturation, e.g., by administering ang-1 or that which stimulates or induces its activity. It also inhibits microvessel development by inhibiting ang-2, which is believed necessary to destabilize a mature vessel, thereby preparing it for new vessel budding and branching. This invention, in its totality, is seen as providing improvements in the therapy, prophylaxis and diagnosis of restenosis and/or atherosclerosis, especially in providing compositions therefor and methods thereof, and thus, the present invention is seen as an advance over the state of the art.

### OBJECTS AND SUMMARY OF THE INVENTION

It is therefore an object of the invention to provide methods and compositions for the diagnosis of, prophylaxis of and/or therapy for restenosis and/or atherosclerosis.

It is yet a further object of the invention to provide such methods and compositions for prophylaxis and/or therapy which comprise an agent for inhibiting VEGF or its activity or its receptors, e.g., the soluble VEGF receptor.

It is yet another object of the invention to provide such methods and compositions for prophylaxis and/or therapy which comprise an agent for inducing vessel maturation, e.g., ang-1 or its activity or its receptors.

It is yet another object of the invention to provide such methods and compositions for prophylaxis and/or therapy which comprise an agent for inhibiting the induction of vessel destabilization (inhibiting the transformation of a mature into an immature vessel), e.g., an inhibitor of ang-2, e.g. ang-1.

It is a still further object of the invention to provide such methods and compositions from in vitro and/or in vivo expression from plasmid DNA, or a vector system, such as a recombinant viral and/or DNA expression system; or from isolation from other sources, or from the administration of the protein itself.

It is a yet further object of the invention to provide such methods and compositions in conjunction with additional treatment methods and compositions.

The present invention thus provides methods and compositions for the diagnosis of, prophylaxis of and/or therapy for restenosis and/or atherosclerosis.

The present invention further provides such methods and compositions for prophylaxis and/or therapy which comprise an agent for inhibiting VEGF or its activity or its receptors, e.g., the soluble VEGF receptor.

The present invention further provides such methods and compositions for prophylaxis and/or therapy which comprise an agent for inhibiting members of the family of FGFs and their signaling cascades.

The present invention also provides such methods and compositions for prophylaxis and/or therapy which comprise an agent for inducing vessel maturation, e.g., ang-1 or its activity or its receptors.

The present invention also provides such methods and compositions for prophylaxis and/or therapy which comprise an agent for inhibiting the induction of vessel destabilization (inhibiting the transformation of a mature into an immature vessel), e.g., an inhibitor of ang-2, e.g. ang-1.

The present invention still further provides such methods and compositions from in vitro and/or in vivo expression from plasmid DNA, or a vector system, such as a recombinant viral and/or DNA expression system; or from isolation from other sources, or from the administration of the protein itself.

The present invention even further provides such methods and compositions in conjunction with additional treatment methods and compositions; note WO 98/33510.

The administration can be after angioplasty, coronary and/or peripheral angioplasty, to prevent the development of, or to provide treatment for, atherosclerosis and/or restenosis. The angioplasty procedure could involve any of the types of angioplasty (e.g. balloon, atherectomy, laser) employed either with or without a stent. Thus, the invention provides a therapeutic method for treatment of atherosclerosis and/or restenosis, and compositions therefor.

Similarly, the compositions of the invention can be administered before, during, or after any type of angioplasty procedure; before angioplasty, to prevent, i.e., as a prophylaxis against, restenosis and/or atherosclerosis. They can also be administered any time during the lifetime of the individual, from childhood to adulthood, to prevent the development or progression of atherosclerosis.

Recombinant viral vectors, such as replication incompetent adenovirus, expressing either or both of the VEGF inhibiting agent or the vessel maturation inducing agent, or expressing an agent that inhibits a vessel destabilizing agent (e.g. inhibits ang-2) can be administered in an amount of about 10⁷ pfu; thus, the inventive compositions can contain, and the inventive methods involve, administering a composition containing recombinant(s), at least this amount; more preferably about 10⁴ pfu to about 10¹⁰ pfu, e.g., about 10⁵ pfu to about 10⁹ pfu, for instance about 10⁶ pfu to about 10⁸ pfu. And, if more than one gene product is expressed by more than one recombinant, each recombinant can be administered in these amounts; or, each recombinant can be administered such that there is, in combination, a sum of recombinants comprising these amounts.

In naked DNA and DNA plasmid compositions, the dosage should be a sufficient amount of naked DNA or DNA plasmid to elicit a response analogous to compositions containing the VEGF inhibiting agent, the vessel maturation agent, or an inhibitor of vessel stabilization, or any combination; or to have expression analogous to dosages in such compositions; or to have expression analogous to expression obtained in vivo by other, e.g., viral, recombinant compositions. For instance, suitable quantities of naked DNA or plasmid DNA in naked DNA or DNA plasmid compositions can be 1 ug to 100 mg, preferably 0.1 to 10 mg, e.g., 500 ug, but lower levels such as 0.1 to 2 mg or even 1-10 ug, may be employed.

And, if more than one gene product is expressed by more than one recombinant and/or DNA (naked or plasmid) system, each recombinant and/or DNA system can be administered in these amounts; or, each recombinant and/or DNA system can be administered such that there is, in combination, a sum of recombinants and/or DNA comprising these amounts.

In protein form, the dosage should be a sufficient amount of naked DNA or DNA plasmid to elicit a response analogous to compositions containing the VEGF inhibiting agent, the vessel maturation agent, or an inhibitor of vessel destabilization, or any combination; or to have amount of protein analogous to dosages in such compositions; or to have amount of protein analogous to expression obtained in vivo by other, e.g., viral, recombinant compositions. For instance, suitable quantities of protein can be 1 ug to 100 mg, preferably 0.1 to 10 mg, e.g., 500 ug, but lower levels such as 0.1 to 2 mg or even 1-10 ug, may be employed.

And, if more than one protein is administered, each protein can be administered in these amounts; or, each protein can be administered such that there is, in combination, a sum of proteins comprising these amounts.

Subcutaneous, intradermal or intramuscular administration are presently preferred. Direct administration to blood vessels (including via catheter-based systems and via by direct intra-arterial infusion) are also encompassed within the invention (see, e.g., Epstein et al., JACC Vol. 23, No. 6, 1994:1278-88(and documents cited therein, incorporated herein by reference); Chang et al., Science 267:518-22(Jan. 27, 1995) (and documents cited therein, incorporated herein by reference)) and; French Patent Application 2723697). The invention further comprehends methods for preparing the compositions of the invention, as well as kits for compositions and methods of the invention. For instance, the invention comprehends a kit comprising an agent for inhibiting VEGF or its receptors or activity, an agent for inducing vessel maturation, and an agent inhibiting vessel destabilization; the agents can be in separate containers; the agents can be in separate containers contained in a package; and, the kit can optionally include instructions for the storage and/or use and/or administration of the agents.

The terms “comprises”, “comprising”, and the like can have the meaning ascribed to them in U.S. Patent Law and can mean “includes”, “including” and the like.

These and other embodiments are disclosed or are obvious from and encompassed by, the following Detailed Description.

### BRIEF DESCRIPTION OF FIGURES

The following Detailed Description, given by way of example, but not intended to limit the invention to specific embodiments described, may be understood in conjunction with the accompanying Figures, incorporated herein by reference, in which:

FIG. 1 shows the microvascular angiogenic processes that occur during restenosis and atherosclerosis; and

FIG. 2 shows the reduction of microangiogenesis by the compositions and methods of the invention.

(With respect to the Figures, reference is made to Suri et al., “Requisite Role of Angiopoietin-1, a Ligand for the TIE2 Receptor, during Embryonic Angiogenesis,” Cell 87:1171-80(December 1996), as the present inventors, as part of the present invention, adapted the accompanying Figures therefrom.)

### DETAILED DESCRIPTION

This invention is designed to employ gene therapy or protein delivery to prevent or treat restenosis, by inhibiting the development of microvessels (vasovasorum) in the injured vessel, insofar as angiogenesis occurring within the vessel wall is an important permissive determinant of neointimal development, and or vessel remodeling. The invention uses various anti-angiogenesis strategies to suppress angiogenesis, and thereby inhibit the development of restenosis and/or atherosclerosis.

Angiogenesis involves the sprouting of capillaries from preexisting blood vessels and/or the development of new vessels. This process is controlled by the action of several angiogenic growth factors and their tyrosine kinase receptors. Two systems involving vascular endothelial growth factor (VEGF) and the angiopoietin-1 ligands, along with their specific receptors (VEGF-R1, VEGF-R2 and Tie-2 respectively), seem to have a unique and specific roll in the induction and maintenance of new blood vessel formation. The angiopoietin-2 ligand also appears to play an important role in the cascade of events leading to angiogenesis. Studies in mice carrying homozygous disruption in these receptors have demonstrated VEGF and angiopoietin-1 act in sequence: 1) VEGF, through VEGF-R1, induces endothelial cell-cell interaction, proliferation, and tube formation; 2) angiopoietin-1, through binding to its receptor Tie-2, elicits recruitment and interaction with peri-endothelial support cells, thus maintaining vessel integrity and stabilizing newly formed blood vessels. Angiopoietin-2 appears to be a functional antagonist of antiopoietin-1; ang-2 expression may be a necessary step in destabilizing an existing vessel, thereby allowing it to initiate new vascular buds and branches. Multiple studies have also demonstrated that members of the family of FGFs and their signaling cascades stimulate angiogenesis (see, e.g., Flugelman et al., Circulation 88(6):2493-500(December 1993)). bFGF and aFGF are in lesions, suggesting that they may play a role in expansion of vasovasorum. It therefore appears that each of these ligands, through their specific receptors, control a specific, complementary function relating to endothelial cells that collectively accounts for a significant part of endothelial cell morphogenesis into mature, functional blood vessels.

An analysis of the many cellular and molecular mechanisms involved in atherogenesis reveals a remarkable parallelism to the cellular and molecular mechanisms involved in restenosis. On these bases, it would appear that strategies designed to inhibit angiogenesis involving the vasovasorum in the patient undergoing angioplasty would, just as in atherosclerosis, also inhibit processes leading to restenosis. The end result would therefore be to inhibit restenosis.

A strategy employed by the present invention is based on the concept that a critical rate-limiting step in restenosis development is the vascular supply of the injured vessel; that neointimal growth, and possibly the amount of negative vascular remodeling, are dependent on the development of a greater number of the blood vessels constituting the vasovasorum, an angiogenic process that can be modulated by anti-angiogenic interventions.

A part of this strategy is based on the concept that most, if not all, therapeutic attempts to inhibit the development of restenosis will carry some immediate or long-term risk. If, however, the “dose” of the intervention could be reduced because of a beneficial effect produced by an anti-angiogenic intervention, then the incidence of side-effects should be substantially diminished. One example of this would be the prevention of restenosis by radiation treatment. Administering anti-angiogenic therapy in conjunction with radiation therapy would, in effect, be a “radiosensitizing” intervention, permitting lower doses of radiation to be administered to achieve the same anti-restenosis effects as achieved by higher radiation doses when administered as single therapy.

The strategy herein has the benefits of substantially reducing the incidence of restenosis with minimal incidence of untoward complications, a result that has been achieved to only a limited extent (or, as with radiation therapy, carrying unknown future risk) with other anti-restenosis strategies.

Although the major intervention strategy of the present invention is to specifically inhibit the molecular cascades of those ligands and/or their receptors that are known to be critically important components of the angiogenesis process, any agent that has anti-angiogenic effects, even if its mechanisms are not currently known, can be used in the practice of the invention. Examples include endostatin, angiostatin, thallidamide, or other agents with broad anti-angiogenic effects. Such other examples include, but are not limited to, agents that inhibit the effects of angiogenic agents, by either binding to the angiogenic agent and preventing its activity, by binding to its receptor, or by inhibiting any aspect of the signaling cascade initiated by the binding of the angiogenic ligand to its receptor. The therapeutic agent could be in the form of a protein, or of a gene which expresses the protein. The gene could be delivered to the patient in a plasmid, or in any other vector, including a viral vector.

Examples of targets for anti-angiogenic strategies include, but need not be limited to VEGF, its receptors, and its signaling cascade, bFGF its receptors, and its signalling cascade; and angiopoeitin-1, its receptor, and its signaling cascade, angiopoeitin-2, its receptor, and its signaling cascade.

Delivery to patient will vary depending on the clinical situation as described in the following situations.

Before, during, or following angioplasty, the anti-angiogenic factor could be administered systemically, either orally or intravenously. It could also be administered directly into the coronary artery in patients undergoing coronary angioplasty, or into the artery supplying the leg in patients undergoing peripheral vessel angioplasty. The anti-angiogenic factor could be applied directly to the wall of the injured vessel via either: 1. a ballon catheter that allows administration of the anti-angiogenic factor directly into the media and/or adventitia, or 2. a stent that has been deployed and which releases the factor into the vessel wall.

Thus, the present invention includes compositions and methods for preventing or treating restenosis and/or atherosclerosis. The present invention includes compositions comprising an agent which inhibits VEGF, e.g., the soluble VEGF receptor, and/or an agent that inhibits angiopoietin-2 (which appears to be a functional antagonist of antiopoietin-1, the expression of which may be a necessary step in destabilizing an existing vessel, thereby allowing it to initiate new vascular buds and branches) and/or an agent which induces vessel maturation, e.g., ang-1; as well as methods comprising the administration of such agent(s), e.g., individually, or separately, or sequentially or the like. That is, the anti-angiogenic factor in the foregoing discussion can be a composition comprising an agent which inhibits VEGF, an agent which inhibits a factor (such as ang-1) causing vessel stabilization and maturation, and an agent which induces vessel maturation. Any or all of these agents can be present in the composition by way of a vector which expresses the agent in vivo.

Angioplasty represents an acute injury model and the present invention is based on findings that many of the processes leading to neointimal development following angioplasty are the same that lead to atherosclerotic plaque development. Studies of cadaver hearts revealed marked development of the vasovasorum of the walls of coronary arteries contiguous to atherosclerotic plaque. During angiogenesis in apoE knockout mice, a considerable number of plaque microvessels were observed in growing atheromata. Administration of endostatin to apoE knockout mice retarded the progression of plague growth, a change associated with a decrease in the amount of microvessels present in the plaque. The inventors have reviewed many specimens of balloon injured porcine coronary arteries and stented porcine coronary arteries and have found that there is a marked angiogenic response involving microvessels of both the adventitia and the neointimal at the site of the vessel injury.

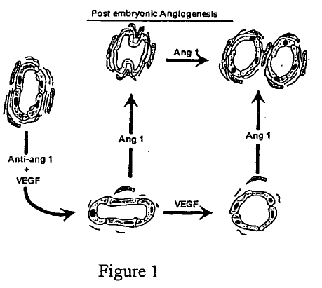

Accordingly, microvascular angiogenesis (expansion of the vasovasorum) occurs during both atherogenesis and during restenosis. The coordinated sequential expression of VEGF and ang-1, and perhaps ang-2, with activation of their signaling cascades, are consistent components of the post embryonic microvascular angiogenic processes that occur during restenosis and atherosclerosis (note for instance FIG. 1: upregulation of VEGF is necessary to destabilize a mature vessel to enable it to begin the angiogenic process, and upregulation of angiopoietin 1 (ang-1) induces vessel maturation).

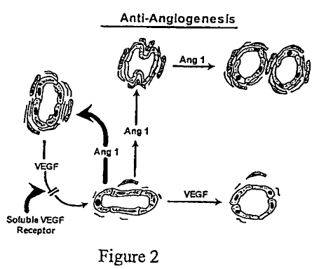

From this, administration of 1) an agent inhibiting VEGF (e.g., the soluble VEGF receptor), and/or 2) an agent inducing vessel maturation (e.g., ang-1 or an agent which induces ang-1), and/or an agent that inhibits ang-2 (ang-2 inhibits ang-1, thereby preventing the vessel stabilization and maturation effects of ang-1) reduces microangiogenesis (FIG. 2) and, thereby, inhibits atherosclerosis (e.g., as shown by the apoe knockout mouse model) and reduces restenosis (e.g., by the porcine coronary artery injury model).

With respect to agents which induce vessel maturation, e.g., ang-1, it is noted that VEGF and angiopoietins, along with their receptors are important regulators (Koblizek et al. Curr Biol 8(9):529-32(April 1998)). Ang-1 and ang-2 modulate VEGF (Asahara et al. Cir Res 83(3):233-40(August 1998)). Ang-2 has been recognized as an antagonist for ang-1 and Tie-2 (Maisonpierre et al. Science 277(5322):55-60(July 1997)). Also, it has been observed that excess soluble Tie-2 abolishes the chemotactic response of endothelial cells towards ang-1; and that ang-2 dose-dependently blocks directed migration towards ang-1, consistent with ang-2 being an inhibitor of ing-1 (Witzenbichler et al. J Biol Chem 273(29):18514-21(July 1998)).

Further, ang-1 has been cloned and plays a mediating role; for instance, mice engineered to lack ang-1 display angiogenic deficits (Suri et al. Cell 87(7):1171-80(December 1996)). Transgenic expression, e.g., overexpression of ang-1 in mice has been demonstrated (Suri et al., Science 282(5388):468-71(October 1998)). And, ang-1 and ang-2 genes have been localized to human genes 8q22.3-q23 and 8p23(Cheung et al. Genomics 48(3):389-91(March 1998)).

Accordingly, using the knowledge of ang-1 having been cloned, that transgenic expression of ang-1 has been demonstrated, and that the ang-1 and ang-2 genes have been localized, to obtain administration of an agent which induces vessel maturation, such as ang-1 or an agent which induces ang-1, no undue experimentation is necessary, as the expression of ang-1 can be performed either in vivo or in vitro; and, one can otherwise obtain ang-1 for administration. Thus, the invention comprehends administration of ang-1 or an agent which stimulates expression or the activity of ang-1.

As to VEGF, it is noted that VEGF induces angiogenesis by binding to VEGF-receptor-2 tyrosine kinase or VEGFR2 TK. The VEGFR2 TK catalytic domain has been cloned and expressed via a baculovirus expression system; Cd2+ was found to be an inhibitor of the enzyme, with inhibition competitive with respect to Mg2+ and non competitive with respect to MgATP (Parast et al. Biochemistry 37(47):16788-801(November 1998); see also Pepper et al. J Cell Physiol 177(3):439-52(December 1998) (by acting in concert bFGF or VEGF, VEGF-C has a potent synergistic effect on the induction of angiogenesis and VEGF, bFGF and VEGF-C are capable of altering endothelial cell extracellular proteolytic activity).

Also, it has been reported that ERK1/2 is necessary for VEGF-induced endothelial cell proliferation; and that MAPK kinase inhibitors abolished ERK1/2 activation in a concentration-dependent manner (Parenti et al. J Biol Chem 272(7):4220-6(February 1998)). 8-(3-oxo-4,5,6-trihydroxy-3h-xanthen-9-yl)-1-naphthoic acid inhibited binding of VEGF to VEGFR-2 or VEGFR-1 as well as MAPK phosphorylation induced by VEGF and could be an inhibitor of VEGF and basic FGF signal transduction (Igarashi et al. Int J Mol Med 2(2):211-215(August 1998)).

Further, VEGF and its receptors (VEGFR-1 and VEGFR-2) as well as ang-1 and its receptor Tie-2 are key transduction systems involved in the regulation of embryonic vascular development; and, inhibition of the VEGF signal transduction resulted in inhibition of neovascularization in angiogenesis-dependent diseases such as proliferative retinopathy or solid tumor growth and the VEGF signal transduction system is useful as anti-angiogenic therapy (Breier et al. Thromb Haemost 78(1):678-83(July 1997); see also Metais et al. Am J. Physiol 275(4 Pt2):H1411-8(October 1998) (effects of coronary artery disease on expression and microvascular response to VEGF)).

In addition, while angiotensin II induced VEGF mRNA production; actinomycin D blocked the induction; and Losartan abolished the induction (Chua et al. Biochim Biophys Acta 1401(2):187-94(February 1998)). Thus, actinomycin D and Losartan may also inhibit VEGF or its activity.

Nonetheless, from the foregoing, it is believed clear that one skilled in the art can select an agent which inhibits VEGF or the activity of VEGF, without any undue experimentation.

An alternative approach to inhibiting VEGF or its activity, can be to inhibit, reduce or diminish the effect or presence of inducers of VEGF or its activity. For instance, VEGF or its expression has been said to be upregulated by glucose deprivation (Satake et al. Biol Cell 90(2):161-8(March 1998)) by Mersalyl, an organomercurial compound (Agani et al. Mol Pharmacol 54(5):749-54(November 1998)) or by H₂O₂ (Chua et al. Free Rad Biol Med 25(8):891-7(November 1998)), and, TNF-alpha has been said to upregulate in a dose and time dependent manner the expression and function of VEGF receptor-2 (Giraudo et al. J Biol Chem 273(34):22128-35(August 1998)).

Thus, to inhibit VEGF, one can inhibit the expression or function of the VEGF receptor, or that which upregulates, e.g., by inhibiting, controlling, modifying, altering, reducing, or diminishing the activity or presence of TNF-alpha. Likewise, one can inhibit VEGF by inhibiting, controlling, modifying, altering, reducing, or diminishing the activity or presence of substances which upregulate or induce VEGF such as glucose, H₂O₂, certain organomercury compounds, and the like. For instance, if glucose deprivation stimulates VEGF activity, then preventing glucose deprivation can be used towards inhibiting VEGF.

Accordingly, the invention comprehends administration of an agent which inhibits VEGF such as an agent which mimics VEGF receptors with respect to binding to VEGF (for instance, an agent which includes a binding region of a VEGF receptor but not regions imparting VEGF receptor activity to the agent) to thereby reduce the amount of VEGF present. The invention also comprehends administration of an agent which mimics VEGF with respect to binding to VEGF receptors, but does not further activate those receptors, e.g., to tie-up the receptors so that VEGF cannot bind to them. The binding envisioned by these agents can be competitive, reversible or irreversible. The invention also comprehends administration of an agent which inhibits VEGF expression or expression of VEGF receptors.

An agent which inhibits VEGF can comprise a plurality of such agents; for instance, agents which bind to different VEGF receptors or which mimic VEGF receptors or which inhibit VEGF and VEGF receptor(s) expression. Thus, combinations of agents which inhibit VEGF are envisioned by the invention.

Likewise, an agent which induces vessel maturation can comprise a plurality of such agents, e.g., ang-1, in combination with an agent which induces ang-1 activity and/or ang-1 expression. And thus, combinations of agents which induce vessel maturation are envisioned by the invention.

As to administration of any or all of the agents inhibiting VEGF or its activity, e.g., soluble VEGF receptor), the vessel maturation inducing agent, and/or the agent inhibiting the vessel maturation inducing agent, these agents can be administered by any suitable means, and such means can include the proteins, naked plasmid DNA, viral vectors, an angioplasty balloon, a catheter-type device that facilitates delivery of the agent(s) to the vessel wall, or intra-arterial infusion (See Witzenbichler et al. Am J Pathol 153(2):381-94(August 1998): VEGF-C promotes angiogenesis; demonstrates administration of VEGF-C by means of naked plasmid DNA (pcVEGF-C 500 microg), polymer coating of an angioplasty balloon (n=8) or as a recombinant human protein (rhVEGF-C 500 microg) by direct intra-arterial infusion; WO 98/33510(vectors including viral vectors, plasmid vectors)).

An agent for inhibiting VEGF or its activity or its receptors and an agent for inducing vessel maturation can be obtained by purification from natural sources or from purification from recombinant sources; and, techniques for such purifications or for protein purification are generally known and require no undue experimentation by the skilled artisan.

The methods for making and/or administering a vector or recombinant for expression of such agents either in vivo or in vitro can be by or analogous to the methods disclosed in: U.S. Pat. Nos. 4,603,112, 4,769,330, 5,174,993, 5,505,941, 5,338,683, 5,494,807, 4,722,848, WO 94/16716, WO 96/39491, Paoletti, “Applications of pox virus vectors to vaccination: An update,” PNAS USA 93:11349-11353, October 1996, Moss, “Genetically engineered poxviruses for recombinant gene expression, vaccination, and safety,” PNAS USA 93:11341-11348, October 1996, Smith et al., U.S. Pat. No. 4,745,051 (recombinant baculovirus), Richardson, C. D. (Editor), Methods in Molecular Biology 39, “Baculovirus Expression Protocols” (1995 Humana Press Inc.), Smith et al., “Production of Huma Beta Interferon in Insect Cells Infected with a Baculovirus Expression Vector,” Molecular and Cellular Biology, December, 1983, Vol. 3, No. 12, p. 2156-2165; Pennock et al., “Strong and Regulated Expression of Escherichia coli B-Galactosidase in Infect Cells with a Baculovirus vector,” Molecular and Cellular Biology March 1984, Vol. 4, No. 3, p. 399-406; EPA 0 370 573, U.S. application Ser. No. 920,197, filed Oct. 16, 1986, EP Patent publication No. 265785, U.S. Pat. No. 4,769,331 (recombinant herpesvirus), Roizman, “The function of herpes simplex virus genes: A primer for genetic engineering of novel vectors,” PNAS USA 93:11307-11312, October 1996, Andreansky et al., “The application of genetically engineered herpes simplex viruses to the treatment of experimental brain tumors,“. PNAS USA 93:11313-11318, October 1996, Robertson et al. “Epstein-Barr virus vectors for gene delivery to B lymphocytes,” PNAS USA 93:11334-11340, October 1996, Frolov et al., “Alphavirus-based expression vectors: Strategies and applications,” PNAS USA 93:11371-11377, October 1996, Kitson et al., J. Virol. 65, 3068-3075, 1991; U.S. Pat. Nos. 5,591,439, 5,552,143 (recombinant adenovirus), Grunhaus et al., 1992, “Adenovirus as cloning vectors,” Seminars in Virology (Vol. 3) p. 237-52, 1993, Ballay et al. EMBO Journal, vol. 4, p. 3861-65, Graham, Tibtech 8, 85-87, April, 1990, Prevec et al., J. Gen Virol. 70, 429-434, PCT WO91/11525, Felgner et al. (1994), J. Biol. Chem. 269, 2550-2561, Science, 259:1745-49, 1993 and McClements et al., “Immunization with DNA vaccines encoding glycoprotein D or glycoprotein B, alone or in combination, induces protective immunity in animal models of herpes simplex virus-2 disease,” PNAS USA 93:11414-11420, October 1996, and U.S. Pat. Nos. 5,591,639, 5,589,466, and 5,580,859 relating to DNA expression vectors, inter alia. See also WO 98/33510; Ju et al., Diabetologia, 41:736-739, 1998(lentiviral expression system); Sanford et al., U.S. Pat. No. 4,945,050(method for transporting substances into living cells and tissues and apparatus therefor); Fischbach et al. (Intracel), WO 90/01543(method for the genetic expression of heterologous proteins by cells transfected); Robinson et al., seminars in IMMUNOLOGY, vol. 9, pp. 271-283(1997) (DNA vaccines); Szoka et al., U.S. Pat. No. 4,394,448(method of inserting DNA into living cells); and McCormick et al., U.S. Pat. No. 5,677,178(use of cytopathic viruses for therapy and prophylaxis of neoplasia).

The expression product generated by vectors or recombinants in this invention can also be isolated from infected or transfected cells and used to prepare compositions for administration to patients.

More generally, compositions for use in the invention can be prepared in accordance with standard techniques well known to those skilled in the pharmaceutical or medical arts. Such compositions can be administered in dosages and by techniques well known to those skilled in the medical arts taking into consideration such factors as the age, sex, weight, and condition of the particular patient, and the route of administration. The compositions can be administered alone, or can be co-administered or sequentially administered with other compositions of the invention or with other prophylactic or therapeutic compositions.

Examples of compositions of the invention include liquid preparations for orifice, e.g., oral, nasal, anal, genital (e.g., vaginal), vascular and/or SMC, etc., administration such as suspensions, syrups or elixirs; and, preparations for parenteral, subcutaneous, intradermal, intramuscular, intravenous, intraarterial (e.g., at site of lesion or plaque), intralymphatic, or intraperitoneal administration (e.g., injectable administration) such as sterile suspensions or emulsions. In such compositions the active agent be in admixture with a suitable carrier, diluent, or excipient such as sterile water, physiological saline, glucose or the like.

The compositions of the invention may be packaged in a single dosage form for immunization by parenteral (i.e., intramuscular, intradermal or subcutaneous) administration or orifice administration, e.g., perlingual (i.e., oral), intragastric, mucosal including intraoral, intraanal, intravaginal, intravenous, intralymphatic, intraarterial (e.g., at site of lesion or plaque), intraperitoneal, and the like administration. Accordingly, compositions in forms for such administration routes are envisioned by the invention. And again, the effective dosage and route of administration are determined by known factors, such as age, sex, weight, condition and nature of patient, as well as LD₅₀ and other screening procedures which are known and do not require undue experimentation.

Dosages of each active agent can range from a few to a few hundred micrograms, e.g., 5 to 500 μg. An inventive vector or recombinant expressing either or both of the VEGF inhibiting agent and/or the vessel maturation inducing agent can be administered in any suitable amount to achieve expression at these dosage levels. The inventive vector or recombinant can be administered to a patient or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu. And, if more than one gene product is expressed by more than one recombinant, each recombinant can be administered in these amounts; or, each recombinant can be administered such that there is, in combination, a sum of recombinants comprising these amounts. Other suitable carriers or diluents can be water or a buffered saline, with or without a preservative. The expression product or isolated product or vector or recombinant may be lyophilized for resuspension at the time of administration or can be in solution.

In plasmid compositions, the dosage should be a sufficient amount of plasmid to elicit a response analogous to compositions wherein the agent or agents are directly present; or to have expression analogous to dosages in such compositions; or to have expression analogous to expression obtained in vivo by recombinant compositions. For instance, suitable quantities of plasmid DNA in plasmid compositions can be 1 ug to 100 mg, preferably 0.1 to 10 mg, e.g., 500 micrograms, but lower levels such as 0.1 to 2 mg or preferably 1-10 ug may be employed. Documents cited herein regarding DNA plasmid vectors may be consulted for the skilled artisan to ascertain other suitable dosages for DNA plasmid vector compositions of the invention, without undue experimentation.

For treatment of restenosis, the compositions comprising the VEGF inhibiting agent and the vessel maturation inducing agent, alone or with other treatment, may be administered as desired by the skilled medical practitioner, from this disclosure and knowledge in the art, e.g., at the first signs or symptoms of restenosis, or as soon thereafter as desired by the skilled medical practitioner, without any undue experimentation required; and, the administration of the compositions, alone or with other treatment, may be continued as a regimen, e.g., monthly, bi-monthly, biannually, annually, or in some other regimen, by the skilled medical practitioner for such time as is necessary to prevent further clogging of blood vessels or further symptoms or signs of restenosis, without any undue experimentation required.

For prevention of restenosis, the compositions, alone or with other treatment, may be administered at the first indication of the patient being prone to restenosis, or as soon thereafter as desired by the skilled medical practitioner, e.g., within six months prior to, immediately prior to, or at angioplasty, such as within six weeks prior to, immediately prior to, or at angioplasty, in any desired regimen such as a single administration or multiple administrations in a regimen as desired, e.g., monthly, bimonthly, biannually, or any combination thereof, without any undue experimentation required. Further, for prevention of restenosis, the compositions, alone or with other treatment, may be administered after or during angioplasty in a regimen of single or multiple administrations as desired by the skilled medical practitioner, such as immediately after, within six weeks after, within six months after, and/or within a year after, e.g., monthly, bi-monthly, biannually, annually, or in some other regimen, by the skilled medical practitioner for such time as is necessary to prevent clogging of blood vessels or symptoms or signs of restenosis, without any undue experimentation required.

For treatment of atherosclerosis, the compositions, alone or with other treatment, may be administered at the first signs or symptoms of atherosclerosis, or as soon thereafter as desired by the skilled medical practitioner, without any undue experimentation required; and, the administration of the compositions, alone or with other treatment, may be continued as a regimen, e.g., monthly, bimonthly, biannually, annually, or in some other regimen, by the skilled medical practitioner for such time as is necessary to prevent further clogging of blood vessels or further symptoms or signs of atherosclerosis, without any undue experimentation required.

For prevention of atherosclerosis, the compositions, alone or with other treatment, may be administered at the first indication of the patient being prone to restenosis and/or atherosclerosis, or as soon thereafter as desired by the skilled medical practitioner, in any desired regimen such as a single administration or multiple administrations in a regimen as desired, e.g., monthly, bi-monthly, biannually, or any combination thereof, without any undue experimentation required, e.g., for such time as is necessary to to prevent clogging of blood vessels or symptoms or signs of atherosclerosis, without any undue experimentation required.

The compositions of the invention can be administered before, during or immediately after the angioplasty to induce maximal responses at the time of angioplasty, since the restenotic process happens quickly.

A better understanding of the present invention and of its many advantages will be had from the following examples, given by way of illustration.

### EXAMPLES

### Example 1

### Atherogenesis

Microvascular angiogenesis (expansion of the vasovasorum) occurs during atherogenesis in the apoE knockout mouse, and the coordinated sequential expression of VEGF and ang-1, with activation of their signaling cascades, are consistent components of the microvascular angiogenic process (see FIG. 1).

The vessels of apoE knockout mice are compared to those of the parental nonatherosclerostic strain.

Endpoint Measurements: To determine whether the VEGF and ang-1 signaling cascades are activated during atherogenesis, vessels are obtained from the parental non-atherosclerotic mice and compared to vessels obtained at various timepoints from apoE knockout mice and analysed for one or more or any or all of:
ang-1 protein (by immunohistochemistry and/or by Western analysis);
tyrosine kinase phosphorylation of TIE 2(to assess the state of activation of the receptor);
VEGF protein (by immunohistochemistry and/or by Western analysis);
tyrosine kinase phosphorylation of one or more of the VEGF receptors (to assess the state of activation of the receptor);
atherosclerotic mass (measured by usual computerized image analysis techniques);
the magnitude of vasovasorum development measured by microscopic CT; and
the magnitude of vasovasorum development measured by immunohistochemistry staining for endothelial cells.

These tests confirm FIG. 1.

Microvascular angiogenesis (expansion of the vasovasorum) is a critical determinant of the degree of atherosclerosis, and the coordinated sequential expression of VEGF and ang-1, and for their receptors, with activation of their signaling cascades, are necessary components of the angiogenic process occurring during atherogenesis. Moreover, a) upregulation of VEGF is necessary to destabilize a mature vessel to enable it to begin the angiogenic process, and b) increased activity of ang-1 without VEGF causes vessel maturation and stabilization, and therefore inhibits ongoing angiogenesis (FIG. 1). Therefore, administration to apoe knockout mice of 1) an agent inhibiting VEGF (e.g., the soluble VEGF receptor) and 2) an agent inducing vessel maturation (e.g., ang-1) will reduce microangiogenesis and, thereby, atherosclerosis and/or restenosis (see FIG. 2).

ApoE knockout mice are treated by intraperitoneal administration of a protein inhibitor of the VEGF pathway (e.g., the soluble VEGF receptor), and with ang-1. These agents are administered as frequently as possible, with the maximal amount determined by the LD50 and by the availability of protein.

Alternatively, the mice are treated by administering into the tail vein a vector or vectors such as adenoviral vector(s) expressing the 1) soluble VEGF receptor transgene, and 2) the ang-1 transgene. It is anticipated that most of the virus is taken up by the liver and protein expression continues for 2-4 weeks. Administrations may be repeated to obtain a desired effect or duration of expression.

Endpoint Measurements: To determine whether the proposed strategy has had biologic effects, vessels are obtained at various timepoints from apoE knockout mice either treated or not treated as indicated and analysed for one or more or any or all of:
ang-1 protein (by immunohistochemistry and/or by Western analysis);
tyrosine kinase phosphorylation of TIE 2 (to assess the state of activation of the receptor);
VEGF protein (by immunohistochemistry and/or by Western analysis);
tyrosine kinase phosphorylation of one or more of the VEGF receptors (to assess the state of activation of the receptor);
atherosclerotic mass (measured by usual computerized image analysis techniques);
the magnitude of vasovasorum development measured by microscopic CT; and
the magnitude of vasovasorum development measured by immunohistochemistry staining for endothelial cells.

The results confirm the foregoing.

### Example 2

### Restenosis

Microangiogenesis (expansion of the vasovasorum) occurs during neointimal development following angioplasty (with or without stents), and the coordinated sequential expression of VEGF and ang-1, with activation of their signaling cascades, are consistent components of the microvascular angiogenic process.

The coronary vessels of pigs are injured by balloon angioplasty with or without stent implantation. To determine whether the VEGF and ang-1 signaling cascades are activated following vessel injury, vessels are obtained from each of 2 pigs sacrificed 2 h, 6 h, 24 h, 14 days and 28 days after injury and analysed.

Endpoint Measurements are one or more or any or all of:
ang-1 protein (by immunohistochemistry and/or by Western blot);
tyrosine kinase phosphorylation of TIE 2(to assess the state of activation of the receptor);
VEGF protein (by immunohistochemistry and/or by Western analysis);
tyrosine kinase phosphorylation of one or more of the VEGF receptors (to assess the state of activation of the receptor);
neointimal mass (measured by usual computerized image analysis techniques);
the magnitude of vasovasorum development measured by microscopic CT; and
the magnitude of vasovasorum development measured by immunohistochemistry staining for endothelial cells.

The results confirm the foregoing.

Microvascular angiogenesis (expansion of the vasovasorum) is a critical determinant of neointimal expansion and therefore of restenosis mass, and the coordinated sequential expression of VEGF and ang-1, and/or their receptors, with activation of their signaling cascades, are necessary components of the restenotic process occurring following vessel injury. Moreover, a) upregulation of VEGF is necessary to destabilize a mature vessel to enable it to begin the angiogenic process, and b) increased activity of ang-1 without VEGF causes vessel maturation and stabilization, and therefore inhibits ongoing angiogenesis (see FIG. 1). Therefore, administration to the injured vessel wall of 1) an agent inhibiting VEGF (e.g., the soluble VEGF receptor) and 2) an agent inducing vessel maturation (e.g., ang-1) will reduce microangiogenesis and, thereby, will reduce neointimal development (see FIG. 2).

Protocol: Following angioplasty, vectors such as adenoviral vectors expressing the 1) soluble VEGF receptor transgene, and 2) the ang-1 transgene are administered into the vessel wall by a balloon catheter that allows injection through multiple small needles of the therapeutic agent directly into the media (e.g., the Infusate catheter (Interventional Technology)).

Endpoint Measurements:

A) Vessels are obtained from each of 2 treated, and each of 2 untreated pigs sacrificed 2 h, 6 h, 24 h, and 14 days after injury and analysed for one or more or any or all of:
ang-1 protein (by immunohistochemistry and/or by Western analysis);
tyrosine kinase phosphorylation of TIE 2 (to assess the state of activation of the receptor);
VEGF protein (by immunohistochemistry and/or by Western analysis); and
tyrosine kinase phosphorylation of one or more of the VEGF receptors (to assess the state of activation of the receptor).

B) Vessels are obtained from each of 8 treated, and each of 8 untreated pigs sacrificed at 28 days after injury and analyzed for one or more or any or all of:
ang-1 protein (by immunohistochemistry and/or by Western analysis);
tyrosine kinase phosphorylation of TIE 2 (to assess the state of activation of the receptor);
VEGF protein (by immunohistochemistry and by Western analysis);
tyrosine kinase phosphorylation of one or more of the VEGF receptors (to assess the state of activation of the receptor);
neointimal mass (measured by usual computerized image analysis techniques);
the magnitude of vasovasorum development measured by microscopic CT; and
the magnitude of vasovasorurn development measured by immunohistochernistry staining for endothelial cells.

Results confirm that administration of a VEGF inhibitor and a vessel maturation inducer prevent or treat atherosclerosis and/or restenosis.

### Example 3

### Formulations and Use

The soluble VEGF receptor, and/or other VEGF inhibitors identified in the foregoing text and ang-1 and/or other vessel maturation inducers are admixed with carrier, diluent etc., as herein described in amounts as herein described to obtain formulations. DNA encoding VEGF inhibitors such as the soluble VEGF receptor and vessel maturation inducers such as ang-1 are used to generate recombinants and DNA expression systems expressing these agents; and, these recombinants and DNA expression systems are admixed with carrier, diluent, etc., as herein described to obtain formulations. Patients are administered the formulations as herein described for the prevention and/or treatment of vascular disease such as atherosclerosis and/or restenosis, including in a manner analogous to gene therapy directed against SMC proliferation, as described in literature cited herein or in documents cited in literature cited herein.

Having thus described in detail preferred embodiments of the present invention, it is to be understood that the invention defined by the appended claims is not to be limited by particular details set forth in the above description as many apparent variations thereof are possible without departing from the spirit or scope thereof.

### REFERENCES

Barger et al., “Hypothesis: vasavasorum and neovascularization of human coronary arteries. A possible role in the pathophysiology of atherosclerosis.” N Eng J Med 310(3): 175-7(January 1984).
Koblizek et al. Curr Biol 8(9):529-32(April 1998).
Witzenbichler et al. J Biol Chem 273(29):18514-21(July 1998).
Asahara et al. Cir Res 83(3):233-40(August 1998).
Suri et al., Science 282(5388):468-71(October 1998).
Cheung et al. Genomics 48(3):389-91(March 1998).
Maisonpierre et al. Science 277(5322):55-60(July 1997).
Suri et al. Cell 87(7):1171-80(December 1996).
Takahashi et al. Jpn J Cancer Res 89(4):445-51(April 1998).
Raymond Presse Med 27(24):1221-4(July 1998).
Folkman, Nature 390:404-7(1997).
Parenti et al. J Biol Chem 272(7):4220-6(February 1998).
Chua et al. Biochim Biophys Acta 1401(2):187-94(February 1998).
Satake et al. Biol Cell 90(2):161-8(March 1998).
Giraudo et al. J Biol Chem 273(34):22128-35(August 1998).
Witzenbichler et al. Am J Pathol 153(2):381-94(August 1998).
Metais et al. Am J. Physiol 275(4 Pt 2):H1411-8(October 1998).
Agani et al. Mol Pharmacol 54(5):749-54(November 1998).
Pepper et al. J Cell Physiol 177(3):439-52(December 1998).
Chua et al. Free Rad Biol Med 25(8):891-7(November 1998).
Parast et al. Biochemistry 37(47):16788-801(November 1998).
Breier et al. Thromb Haemost 78(1):678-83(July 1997).
Igarashi et al. Int J Mol Med 2(2):211-215(August 1998). WO 98/33510.
Kwon et al., Journal of Clinical Investigation, 101(8): 1551-56(April 1998).
Kwon et al., “Adventitial Vasa Vasorum in Balloon-injured Coronary Arteries: Visualization and Quantitation by a Microscopic Three-dimensional Computed Tomography Technique” (1998).
Inoue et al., Circulation 98:2108-2116 (November 1998).
Marx, Science, Vol. 265 page 320 (Jul. 15, 1994).
Epstein et al., JACC Vol. 23, No. 6, 1994:1278-88.
Flugelman et al., “Smooth muscle cell abundance and fibroblast growth factors in coronary lesions of patients with nonfatal unstable angina. A clue to the mechanism of transformation from the stable to the unstable clinical state.” Circulation 88(6):2493-500(December 1993).
Change et al., Science 267:518-22 (Jan. 17, 1995).
French Patent Application 2723697.

What is claimed is:

1. A composition for therapy for restenosis and/or atherosclerosis comprising an agent for inhibiting VEGF (VEGF inhibitor) and an agent for inducing vessel maturation (vessel maturation inducer).

2. The composition of claim 1 wherein at least one of the VEGF inhibitor and the vessel maturation inducer comprises an expression system which expresses at least one of the VEGF inhibitor and the vessel maturation inducer.

3. The composition of claim 1 wherein the VEGF inhibitor comprises the soluble VEGF receptor.

4. The composition of claim 1 wherein the vessel maturation inducer comprises ang-1.

5. The composition of claim 3 wherein the vessel maturation inducer comprises ang-1.

6. The composition of claim 2 wherein the expression system comprises at least one recombinant.

7. The composition of claim 6 wherein the recombinant is an adenovirus, poxvirus, baculovirus, or DNA plasmid expression system.

8. A method for preventing or treating atherosclerosis or restenosis comprising administering a composition as claimed in claim 1.

9. A kit for preventing or treating atherosclerosis or restenosis as claimed in claim 8 comprising an agent for inhibiting VEGF (VEGF inhibitor) and an agent for inducing vessel maturation (vessel maturation inducer).

10. The kit of claim 9 wherein the VEGF inhibitor and the vessel maturation are in separate containers.

11. The kit of claim 10 wherein the separate containers are in a package together.
